(19) United States
(12) Patent Application Publication
(10) Pub. No.: US 2025/0041120 A1
(43) Pub. Date: Feb. 6, 2025

(54) METHOD FOR OPTIMIZING LASER PARAMETERS FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/787,387

(22) Filed: Jul. 29, 2024

(30) Foreign Application Priority Data
Aug. 3, 2023 (DE) 10 2023 120 645.1

(51) Int. Cl. A61F 9/008

(52) U.S. Cl. CPC A61F 9/00827; A61F 2009/00872

(57) ABSTRACT

The invention relates to apparatuses and methods for optimizing laser parameters for an ophthalmological laser (12) of a treatment apparatus (10). The method comprises determining (S12) if a treatment quality with the treatment apparatus, which has been performed with first laser parameters (S10), corresponds to a preset treatment quality criterion, wherein the laser parameters include at least a laser pulse energy, a spatial laser pulse distance and a spatial laser pulse path distance; if the treatment quality does not correspond to the treatment quality criterion, providing second laser parameters (S14), in which at least one laser parameter is adapted by a preset value; and wherein the second laser parameters are used for a subsequent treatment with the treatment apparatus (S16).

### FIELD

The invention relates to a method for optimizing laser parameters for an ophthalmological laser of a treatment apparatus. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus situated within the organic tissue to remove a tissue, in particular a tissue lenticule, from the cornea.

In treatment apparatuses, the laser pulse energy, a spatial laser pulse distance in the cornea and a spatial laser pulse path distance between adjacent laser pulse paths in the cornea are usually freely adjustable. Therein, the quality of the performed incisions and/or ablations of corneal tissue depends on the suitable choice of these laser parameters. However, the suitable choice of these laser parameters and thereby the quality of the incisions can additionally be dependent on the location of the treatment apparatus, in particular on environmental conditions, and/or further parameters. Thus, it is often not apparent, which laser parameters are most suitable for the respective treatment apparatus and/or if the laser parameters have to be adapted over time.

### SUMMARY

It is the object of the invention to optimize laser parameters for an ophthalmological laser of a treatment apparatus.

This object is solved by the independent claims. Advantageous embodiments of the invention are disclosed in the dependent claims, in the following description as well as the figures.

The invention is based on the idea that at least one treatment is performed with the treatment apparatus, preferably multiple treatments are performed, wherein a treatment quality or a treatment result is automatically or manually restored to correspondingly adapt the laser parameters. This means that a regulation of the laser parameters can be provided depending on at least one previously performed treatment.

An aspect of the invention relates to a method for optimizing laser parameters for an ophthalmological laser of a treatment apparatus, wherein the method comprises the following steps performed by a control device. Therein, an appliance or an appliance component, in particular a computer or processor, which can automatically or semi-automatically perform the following steps, is to be understood by a control device: determining if a treatment quality of a treatment, which has been performed with first laser parameters, corresponds to a preset treatment quality criterion, wherein the laser parameters include at least a laser pulse energy, a spatial laser pulse distance and a spatial laser pulse path distance; if the treatment quality does not correspond to the treatment quality criterion, providing second laser parameters, in which at least one laser parameter is adapted by a preset value, in particular depending on the treatment quality criterion, wherein the second laser parameters are used for a subsequent treatment with the treatment apparatus.

In other words, a treatment with the treatment apparatus can first be performed, which comprises first laser parameters. Herein, the laser parameters can include at least a laser pulse energy, a distance between adjacent laser pulses and a distance between adjacent laser pulse paths. For example, the treatment can be performed within the scope of a refraction correction of a cornea of a patient.

After performing this treatment, it can be examined if a treatment quality corresponds to a preset treatment quality criterion. The treatment quality can for example be automatically determined by a capturing device, for example by a camera, or the treatment quality can be ascertained by a user, in particular a physician, who can provide results of an assessment to the control device. The treatment quality can for example include an incision quality, this means a success of a tissue separation, a development of an opaque bubble layer, which in particular occurs with a too high energy deposition in the tissue, or the development of black spots, which occur with a too low energy deposition in the tissue. Thus, various aspects of the treatment quality can be assessed by the treatment quality criterion, whereby it can be decided whether or not the laser parameters are to be adapted. The treatment quality criterion can be a condition or rule, for which the treatment quality is examined.

If it is determined that the treatment quality does not correspond to the treatment quality criterion, the first laser parameters can be adapted, whereby second laser parameters are provided. For the second laser parameters, at least one laser parameter of the first laser parameters can be changed by a preset value, in particular depending on the treatment quality and/or the treatment quality criterion. This means that either the laser pulse energy, the spatial pulse distance or the spatial laser pulse path distance or multiple of the previously mentioned laser parameters can be changed. Herein, it is particularly preferably provided that they are changed by a preset value, which is preset such that the corresponding laser parameters change in small steps to the effect that the treatment quality criterion is satisfied. In some implementations, the second laser parameters, then, can be the same as the first laser parameters but for the laser parameters which are changed by a preset value.

In particular, the second laser parameters can be used for a subsequent treatment with the treatment apparatus. This means that the treatment apparatus can be controlled with the second laser parameters by the control device. Preferably, the method can be iteratively performed, which means that after providing the second laser parameters, it can be again examined if the treatment quality corresponds to the treatment quality criterion, wherein the second laser pulse parameters can subsequently be again adapted. This means that the second laser parameters become the first laser parameters in a repetition of the method, and it can be again examined if the treatment quality criterion is present.

By the invention, the advantage arises that the laser parameters can be continuously optimized, in particular independently of a location and/or environmental conditions of the treatment apparatus.

The invention also includes embodiments, by which additional advantages arise.

In an embodiment the treatment quality criterion includes at least one of the following aspects: a development of an opaque bubble layer in a treatment area, a development of black spots in the treatment area, a separation result, which is in particular determined via a required dissection time of irradiated tissue, and/or a convalescence result, which is in particular ascertained via an inflammatory reaction and/or via an achieved visual acuity. In other words, it can be examined by the treatment criterion if an opaque bubble layer develops in a treatment area. An opaque bubble layer can occur if a too high energy deposition occurs in a tissue of the treatment area, since excessive energy enters the tissue and structurally changes and blurs it. For example, this can be determined by a capturing device, in particular a camera. Alternatively or additionally, it can be examined if black spots develop in the treatment area. Black sports can for example develop if an energy deposition in the tissue of the treatment area is not sufficient to separate the tissue, whereby the tissue is changed and changes color to black. The development of black spots can also be ascertained by a capturing device. Alternatively or additionally, a separation result can be examined by the treatment quality criterion, thus a quality of the incision performed by the laser in the treatment area. Hereto, it can in particular be ascertained how fast the irradiated tissue detaches or can be detached from surrounding cornea, wherein this can be provided as the dissection time. Alternatively or additionally, a convalescence result can be examined by the treatment quality criterion. This means that it can be examined how well a patient tolerates a treatment with the treatment apparatus, which has the first laser parameters. Hereto, presence of an inflammatory reaction in the eye or the cornea can in particular be ascertained, for example by a physician, who can input the results into the control device. An achieved visual acuity, this means vision restrictions after the treatment, can for example also be used for determining the convalescence result. In other words, it can be ascertained by the convalescence result how fast an eye recovers after the treatment.

In another embodiment the treatment quality criterion includes a development of an opaque bubble layer in a treatment area, wherein upon development of the opaque bubble layer, for providing the second laser parameters for the subsequent treatment, the laser pulse energy is reduced and/or wherein the spatial pulse distance and/or the spatial laser pulse path distance are increased. In other words, if an opaque bubble layer develops in the treatment area in using the first laser parameters, the laser pulse parameters can be adapted such that the result of the formula [laser pulse energy/(laser pulse distance*laser pulse path distance)] is reduced. Hereby, the advantage arises that an opaque bubble layer can be avoided or reduced for a subsequent treatment.

In another embodiment the treatment quality criterion includes a development of an opaque bubble layer in a treatment area, wherein upon non-appearance of the opaque bubble layer, for providing the second laser parameters for the subsequent treatment, the laser pulse energy is increased and/or wherein the spatial laser pulse distance and/or the spatial laser pulse path distance are reduced. In other words, if an opaque bubble layer is not generated by the first laser parameters, the energy deposition in the treatment area can be increased to for example achieve an improved separation result. In particular, the result of the formula [laser pulse energy/(laser pulse distance*laser pulse path distance)] can be increased. Hereby, the advantage arises that an optimized limit value between a development of an opaque bubble layer and an improved separation result can be iteratively approximated.

In another embodiment the treatment quality criterion includes a development of black spots in a treatment area, wherein upon development of the black spots, for providing the second laser parameters for the subsequent treatment, the laser pulse energy is increased and/or wherein the spatial laser pulse distance and/or the spatial laser pulse path distance are reduced. In other words, the development of black spots can be examined by the treatment quality criterion, wherein an energy deposition in the treatment area is increased if black spots develop. In particular, the result of the formula [laser pulse energy/(laser pulse distance*laser pulse path distance)] can be increased. Hereby, the advantage arises that a development of black spots can be avoided or reduced.

In particular, it is provided that the development of the opaque bubble layer and/or of the black spots in the treatment area is ascertained by a camera device. This means that the treatment apparatus for example can use a camera device, which includes one or more cameras for monitoring the treatment area, to capture a change of the tissue, in particular a development of an opaque bubble layer and/or of black spots. The camera can for example record data in the visual or infrared spectral range. Hereby, the advantage arises that the treatment quality criterion can be monitored with the treatment apparatus during a treatment.

In another embodiment the treatment quality criterion includes a separation result, wherein, if the separation result is assessed as deficient, for providing the second laser parameters for the subsequent treatment, the laser pulse energy is increased and/or wherein the spatial laser pulse distance and/or the spatial laser pulse path distance are reduced. In other words, a separation result can be examined by the treatment quality criterion. Herein, the separation result can for example be assessed according to a preset scale, which provides at least a differentiation between a deficient and a sufficient or good treatment. Preferably, further assessment stages, for example in the form of a school grade system, can be provided. If the separation result is assessed as deficient, an energy deposition in the tissue of the treatment area can be increased for a subsequent treatment. In particular, a result of the formula [laser pulse energy/(laser pulse distance*laser pulse path distance)] can be increased. Preferably, the separation result can be input by an input device by a user, for example a physician, who can examine and assess the separation result. Hereby, the advantage arises that a separation of a tissue can be continuously adapted and improved.

In another embodiment the treatment quality criterion includes a convalescence result, wherein, if the convalescence result is assessed as deficient, for providing the second laser parameters for the subsequent treatment, the laser pulse energy is reduced and/or wherein the spatial laser pulse distance and/or the spatial laser pulse path distance are increased. This means that a convalescence result or a patient recovery can be examined by the treatment quality criterion, wherein—if it is deficient—an energy deposition in the tissue of the treatment area can be reduced. In particular, a result of the formula [laser pulse energy/(laser pulse distance*laser pulse path distance)] can be reduced. Preferably, the convalescence result can be input by an input device by a user, for example a physician, who can examine and assess the convalescence result. Hereby, the advantage arises that a treatment can be continuously adapted and improved.

In another embodiment a degree is determined for the respective aspect of the treatment quality criterion, wherein the respective aspect is categorized into a severe, medium and slight degree, wherein all of the three laser parameters are adapted in case of a severe degree, wherein two laser parameters are adapted in case of a medium degree, and wherein one of the laser parameters is adapted in case of a slight degree. In other words, a categorization can occur for the respective aspects of the treatment quality criterion, how severely the aspect is affected. Herein, one of the previously mentioned characteristics is meant by aspect of the treatment quality criterion, thus, one aspect of the treatment quality criterion can for example include the development of the opaque bubble layer, the development of the black spots, the separation result and/or the convalescence result. They can each be categorized into one of at least three preset degrees, in particular a severe, medium and slight degree. For example, it can be examined in case of an opaque bubble layer how many opaque areas develop in a cornea, wherein the degree can then be categorized hereby. Thus, depending on the degree of the opaque bubble layer, of the black spots, of the separation result and/or of the patient recovery, one to three laser parameters, in particular the laser pulse energy, the spatial laser pulse distance and/or the spatial laser pulse path distance, can be correspondingly adapted. Thus, in case of an opaque bubble layer, for which a severe degree has been ascertained, a laser pulse energy can for example be reduced by 5 nJ, a laser pulse distance can be increased by 0.1 μm and a laser pulse path distance can be increased by 0.2 μm, and in case of a medium degree, the laser pulse energy can be reduced by 5 nJ and a laser pulse path distance can for example be increased by 0.2 μm, and in case of a slight degree, it can be provided that only the laser pulse energy is reduced by 5 nJ. Hereby, the advantage arises that optimized laser parameters can be faster achieved according to degree of the impairment.

In another embodiment the laser pulse energy is adapted with a preset value of 5 nJ and/or the spatial laser pulse distance is adapted with a preset value of 0.1 μm and/or the spatial laser pulse path distance is adapted with a preset value of 0.2 μm. Herein, “adapt” means that the respective laser parameter can be increased or reduced by this value depending on which treatment quality criterion is not present. These preset values represent suitable possibilities of adjustment, in particular for an iterative adjustment of the laser parameters, since a change is thus provided, but which is not too high to generate other impairing effects.

In another embodiment limits are preset for the respective laser parameter, which are not exceeded by the adaptation of the laser parameters, in particular 60 nJ as the lower limit and 300 nJ as the upper limit for the laser pulse energy and 1.5 μm as the lower limit and 18 μm as the upper limit for the spatial laser pulse distance and 0.5 μm as the lower limit and 10 μm as the upper limit for the spatial laser pulse path distance. In other words, the ranges, in which the laser parameters can be changed, can be restricted by preset limits. Herein, the preset limits can be preset such that the laser parameters do not fall outside of safety regulations or limits of the treatment apparatus.

In another embodiment the laser parameters are adapted depending on a treatment location, at which an impairment of the treatment quality has been determined. In other words, the laser parameters can be differently adapted for different treatment locations or treatment positions in the cornea. Thus, the laser parameters can for example only be adapted in a peripheral area of a treatment, or in a center. Thus, laser parameters, which are overall optimized to the treatment, can for example only be adapted where an impairment arises. This can in particular occur upon development of an opaque bubble layer, in which a development can in particular occur at the beginning of a treatment in a peripheral area, wherein after generation of the first cavitation bubble path, sufficient space for dissipating excessive energy is provided, such that other laser parameters are possible in more central treatment locations. Hereby, the advantage arises that a treatment result can be improved.

In another embodiment the treatment quality is examined for a preset number of treatments with the first laser parameters before the second laser parameters are provided. In other words, the adaptation of the laser parameters is not performed after each treatment, but after a preset number of treatments. In particular, it can be provided that the adaptation of the laser parameters is performed after at least two, preferably multiple treatments, for example five or ten treatments. Herein, statistics from all of the previously performed treatments can particularly preferably also be formed to examine the treatment quality by the treatment quality criterion. Hereby, the advantage arises that a further improvement and optimization of the laser parameters can be provided.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided second laser parameters to at least one ophthalmological laser of the treatment apparatus and controlling the treatment apparatus and/or the laser with the second laser parameters and/or by control data. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by optical breakdown, in particular at least partially separate it by photodisruption and/or to ablate corneal layers by (photo) ablation and/or to effect a laser-induced refractive index change in the cornea and/or the eye lens and/or to increase a crosslinking of the cornea.

In another embodiment of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown can be spatially narrowly limited such that a high incision accuracy is allowed in the generation of the interfaces. In particular, the range between 700 nm and 780 nm can also be selected as the wavelength range.

In another embodiment of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram according to an exemplary embodiment.

### DETAILED DESCRIPTION

In the figures, identical or functionally identical elements are provided with the same reference characters.

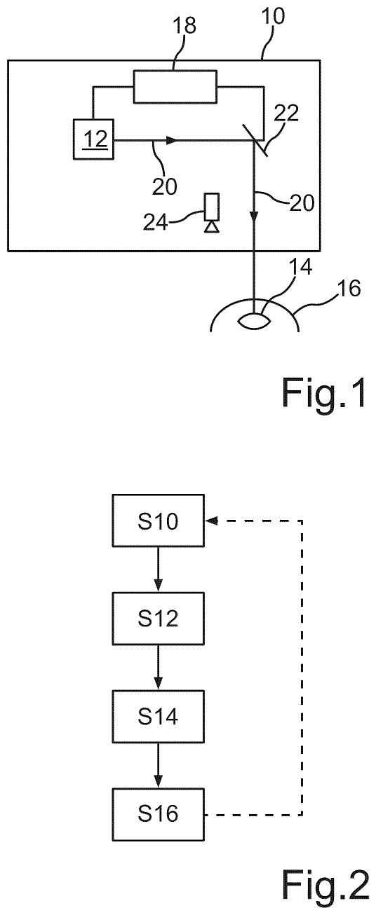

FIG. 1 shows a schematic representation of a treatment apparatus 10 with an ophthalmological laser 12 for removing a tissue 14 from a human or animal cornea 16 by photodisruption and/or ablation. For example, the tissue 14 can represent a lenticule or also volume body, which can be separated from the cornea 16 by the eye surgical laser 12 for correcting a visual disorder. A correction profile or a geometry of the tissue 14 to be removed can be provided by a control device 18, in particular in the form of control data, such that the laser 12 emits pulsed laser pulses in a pattern predefined by the control data into the cornea 16 of the eye to remove the tissue 14. Hereto, laser parameters can further be provided by the control device 18, which define at least a laser pulse energy, a spatial laser pulse distance and a spatial laser pulse path distance. Alternatively, the control device 18 can be a control device 18 external with respect to the treatment apparatus 10.

Furthermore, FIG. 1 shows that the laser beam 20 generated by the laser 12 can be deflected towards the cornea 16 by a beam deflection device 22 such as for example a rotation scanner, to remove the tissue 14. The beam deflection device 22 can also be controlled by the control device 18 to remove the tissue 14.

Preferably, the illustrated laser 12 can be a photodisruptive and/or photoablative laser, which is formed to emit laser pulses in a wavelength range between 300 nanometers and 1400 nanometers, preferably between 700 nanometers and 1200 nanometers, at a respective pulse duration between 1 femtosecond and 1 nanosecond, preferably between 10 femtoseconds and 10 picoseconds, and a repetition frequency of greater than 10 kilohertz, preferably between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

Furthermore, FIG. 1 shows a camera device 24, which can be formed to monitor a treatment area of the cornea 16. The camera device 24 can for example be part of the treatment apparatus 10 or be provided separately therefrom. The data, which can be recorded by the camera device 24, can be provided to the control device 18, in particular for examining a treatment quality.

For example, the examination of the treatment quality can be used to suitably adapt or optimize laser parameters of the treatment apparatus 10 to improve a treatment result. Thereto, the method shown in FIG. 2 can be performed.

FIG. 2 shows a method diagram for optimizing laser parameters for an ophthalmological laser 12 of a treatment apparatus 10. In a step S10, a treatment can be performed with the treatment apparatus 10, wherein the laser 12 and/or the beam deflection device 22 can be controlled with first laser parameters, which include at least a laser pulse energy, a spatial laser pulse distance and a spatial laser pulse path distance.

In a step S12, a treatment quality of the treatment, which has been performed with the first laser parameters, can be examined thereafter. Hereto, the performed incisions in the cornea 16 for separating the tissue 14 can for example be captured by a camera device 24, wherein they can be subsequently inspected by the control device 18 for presence of one or more treatment quality criteria. Alternatively or additionally, the treatment quality criterion can also be examined by a user, for example a physician, wherein results of this examination, such as for example an assessment of the incisions and/or a convalescence of the patient or of the cornea 16, can be provided to the control device 18 via a user interface (not shown).

The treatment quality criterion, which can be examined for examining the treatment quality, can include at least one, preferably multiple, of the following aspects. A development of an opaque bubble layer in the cornea 16 can be examined, a development of black spots in the cornea 16 can be examined, a separation result of incision surfaces of the volume body 14 can be examined and/or a convalescence result, that is for example inflammatory reactions after the treatment, can be examined.

In a step S14, the first laser parameters can be adapted to second laser parameters, in which at least the laser pulse energy and/or the spatial laser pulse distance and/or the spatial laser pulse path distance can be adapted by a preset value compared to the first laser parameters, if the treatment quality does not correspond to the treatment quality criterion.

For example, it can be determined that an opaque bubble layer develops in the cornea, wherein the laser pulse energy can be reduced in this case. Alternatively or additionally, the spatial laser pulse distance and/or the spatial laser pulse path distance can be increased. In contrast, if an opaque bubble layer does not develop and the treatment quality still does not correspond to the treatment quality criterion, the laser pulse energy can be increased and/or the distances can be reduced to improve a tissue separation.

Alternatively or additionally, it can be examined if black spots develop upon irradiation with the first laser parameters in the treatment area of the cornea 16, wherein the laser pulse energy can be increased and/or the laser pulse distances can be reduced in this case.

Alternatively or additionally, a separation result can be examined, for example whether or not the volume body 14 can be removed from the cornea 16 without difficulties, wherein, if the separation result is assessed as deficient, the laser pulse energy can be increased and/or the spatial laser pulse distance and the spatial laser pulse path distance, respectively, can be reduced for a subsequent treatment.

In particular, it can be provided that for increasing or reducing the laser parameters for providing the second laser parameters, a laser pulse energy is increased or reduced by a preset value of 5 nJ and/or the spatial laser pulse distance is increased or reduced by a preset value of 0.1 μm and/or the spatial laser pulse path distance is increased or reduced by a preset value of 0.2 μm. Herein, it can preferably be provided that the values are not adapted below 60 nJ and above 300 nJ for the laser pulse energy, the laser pulse distance is not adapted below 1.5 μm and above 18 μm and the laser pulse path distance is not adapted below 0.5 μm and above 10 μm.

Furthermore, it can be assessed by the treatment quality criterion how great or which degree one of the above mentioned effects has in the cornea 16. Preferably, they can be divided at least according to a severe, medium and slight degree. If it is determined that a severe degree is present, the laser pulse energy, the spatial laser pulse distance and the spatial laser pulse path distance can be adapted at the same time. In case of a medium degree, two laser parameters can be adapted, and in case of a slight degree, only one of the laser parameters can be adapted.

In a step S16, the second laser parameters can finally be adjusted for a subsequent treatment with the treatment apparatus 10 and the laser can be controlled with the second laser parameters. Preferably, it can be provided that the method can subsequently be iteratively repeated, which is indicated by the dashed line between step S16 and step S10. Thus, the laser parameters can be continuously optimized. Preferably, it can be provided that the laser parameters are not adjusted after each treatment, but it can be examined only after a preset number of treatments if an adaptation is required, wherein statistics can in particular be created from the performed treatments to examine the presence of the treatment quality criterion.

Overall, the examples show, how an automatic optimization of laser parameters can be achieved.

1. A method for optimizing laser parameters for an ophthalmological laser of a treatment apparatus, wherein the method comprises the following steps performed by a control device:
determining if a treatment quality of a treatment, which has been performed with first laser parameters, corresponds to a preset treatment quality criterion, wherein the laser parameters include at least a laser pulse energy, a spatial laser pulse distance and a spatial laser pulse path distance;
if the treatment quality does not correspond to the treatment quality criterion, providing second laser parameters, in which at least one of the laser parameters is adapted by a preset value;
wherein the second laser parameters are used for a subsequent treatment with the treatment apparatus.

2. The method according to claim 1, wherein the treatment quality criterion includes at least one of the following aspects:
a development of an opaque bubble layer in a treatment area;
a development of black spots in the treatment area;
a separation result, which is determined via a required dissection time of irradiated tissue;
a convalescence result, which is ascertained via an inflammatory reaction and/or an achieved vision.

3. The method according to claim 1, wherein the treatment quality criterion includes a development of an opaque bubble layer in a treatment area, wherein upon development of the opaque bubble layer in the treatment area, for providing the second laser parameters for the subsequent treatment, the laser pulse energy is reduced and/or wherein the spatial laser pulse distance and/or the spatial laser pulse path distance are increased.

4. The method according to claim 1, wherein the treatment quality criterion includes a development of an opaque bubble layer in a treatment area, wherein upon non-appearance of the opaque bubble layer in the treatment area, for providing the second laser parameters for the subsequent treatment, the laser pulse energy is increased and/or wherein the spatial laser pulse distance and/or the spatial laser pulse path distance are reduced.

5. The method according to claim 1, wherein the treatment quality criterion includes a development of black spots in a treatment area, wherein upon development of the black spots in the treatment area, for providing the second laser parameters for the subsequent treatment, the laser pulse energy is increased and/or wherein the spatial laser pulse distance and/or the spatial laser pulse path distance are reduced.

6. The method according to claim 2, wherein the development of the opaque bubble layer and/or of the black spots in the treatment area is ascertained by a camera device.

7. The method according to claim 1, wherein the treatment quality criterion includes a separation result, wherein, if the separation result is assessed as deficient in the treatment area, for providing the second laser parameters for the subsequent treatment, the laser pulse energy is increased and/or wherein the spatial laser pulse distance and/or the spatial laser pulse path distance are reduced.

8. The method according to claim 1, wherein the treatment quality criterion includes a convalescence result, wherein if the convalescence result is assessed as deficient in the treatment area, for providing the second laser parameters for the subsequent treatment, the laser pulse energy is reduced and/or wherein the spatial laser pulse distance and/or the spatial laser pulse path distance are increased.

9. The method according to claim 2, wherein a degree is determined for a respective aspect of the treatment quality criterion, wherein the respective aspect is categorized into a severe, medium and slight degree, wherein three laser parameters are adapted in case of severe degree, wherein two laser parameters are adapted in case of medium degree, and wherein one of the laser parameters is adapted in case of slight degree.

10. The method according to claim 1, wherein the laser pulse energy is adapted with a preset value of 5 nJ and/or the spatial laser pulse distance is adapted with a preset value of 0.1 μm and/or the spatial laser pulse path distance is adapted with a preset value of 0.2 μm.

11. The method according to claim 1, wherein preset limits are preset for the laser parameters, which are not exceeded by the adaptation of the laser parameters.

12. The method according to claim 1, wherein the laser parameters are adapted depending on a treatment location, at which an impairment of the treatment quality has been determined.

13. The method according to claim 1, wherein the treatment quality is examined for a preset number of treatments with the first laser parameters before the second laser parameters are provided.

14. A control device, which is configured to perform a method according to claim 1.

15. A treatment apparatus with at least one ophthalmological laser for separation of a corneal volume with predefined interfaces of a human or animal eye by optical breakdown and at least one control device according to claim 14.

16. (canceled)

17. A computer-readable medium for storing a computer program, the computer program comprising commands which cause a treatment apparatus to execute a method according to claim 1.

18. The method according to claim 11, wherein the preset limits comprise a lower limit and an upper limit, wherein for the laser pulse energy, 60 nJ is preset as the lower limit and 300 nJ is preset as the upper limit; and for the spatial pulse distance, 1.5 μm is preset as the lower limit and 18 μm is preset as the upper limit; and for the spatial laser pulse path distance, 0.5 μm is preset as the lower limit and 10 μm is preset as the upper limit.
